(19) United States
(12) Patent Application Publication
(10) Pub. No.: US 2019/0382727 A1
(43) Pub. Date: Dec. 19, 2019

(54) CULTURE MEDIA FOR SIMULTANEOUS TESTING OF MULTIPLE HEMATOPOIETIC DISEASE TYPES

(71) Applicant: Precipio, Inc., New Haven, CT (US)

(72) Inventor: Ayman MOHAMED, East Haven, CT (US)

(21) Appl. No.: 16/438,507

(22) Filed: Jun. 12, 2019

Related U.S. Application Data
(60) Provisional application No. 62/685,526, filed on Jun. 15, 2018.

(51) Int. Cl. C12N 5/0775; G01N 33/487; C12N 5/0789; C12Q 1/6827; C12N 5/0781; C12N 5/0783

(52) U.S. Cl. CPC C12N 5/0663; G01N 33/48735; C12N 5/0647; C12Q 1/6827; C12N 5/0635; C12N 5/0636; C12N 2502/1107; C12N 2501/10; C12N 2500/32; C12N 2500/84; C12N 2501/999; C12N 2501/998; C12N 2502/1171; C12N 2501/052

(57) ABSTRACT

The present disclosure provides, in part, a culture medium comprising: interleukin-2; lipopolysaccharide; one, two or more antibiotics; animal serum; and a growth factor, and methods for using same.

### BACKGROUND

Hematopoietic malignancies are a leading cause of death in the US and are cancers that derive from either of the two major blood cell lineages: myeloid and lymphoid cell lines. Hematopoietic malignancies are characterized by abnormal and neoplastic proliferation of blood cells and include, for e.g., Acute Myeloid Leukemia, Acute Promyelocytic Leukemia, Chronic Myeloid Leukemia, Acute Monocytic Leukemia, Acute Megakaryoblastic Leukemia, Acute Lymphoblastic Leukemia, Chronic Lymphocytic Leukemia, T Cell Lymphoma and, Multiple Myeloma.

Historically, the first step in diagnosis of a hematopoietic malignancy was based primarily on morphological analysis of peripheral blood cells. More recently, morphological analysis has been supplemented with chromosomal or karyotype analysis to identify specific genomic abnormalities. This has led to breakthroughs in the diagnosis and treatment of several hematopoietic malignancies, particularly chronic myeloid leukemia and acute myeloid leukemia with translocation (15:17). Hence, chromosomal analysis of hematopoietic cells for diagnosis of malignancies is a rapidly growing area of clinical cytogenetics.

As part of the process to diagnose hematopoietic diseases, various cytogenetic tests are required. In these tests, different cells are cultured in vitro in various ways to imitate the cell's behavior within the body. Generally, there are four groups of cells that can be investigated as part of the case workup for a patient presenting with symptoms: Myeloid cell lines—indicating myeloid disease disorders, B-cells—indicating B-cell disorders, T-cells—indicating T-cell disorders and Plasma cells—indicating plasma cell disorders.

Currently, a cytogeneticist must decide a priori which cell type should be cultured using a patient's cell sample, e.g., from a bone marrow biopsy. In most cases, due to either specimen limitation or cell viability, the cytogeneticist typically selects only one of the aforementioned cell lines to culture. In vitro culturing of the selected cell lineage from bone marrow or peripheral blood may necessitate supplementation of the culture medium with particular mitogens, growth factors or combinations thereof, which may be a unique requirement for that cell type. The consequence of this approach is that it severely limits flexibility; it is not possible to change the preferential selection (or growth) of one cell lineage to another. Hence, if the wrong cell line is selected, the diagnosis may be compromised or generate a false negative result because the wrong cell lineage may have been investigated. Further, this current approach can delay diagnosis and/or the cell sample viability if another cell lineage should be cultured to confirm a specific hematopoietic malignancy.

Accordingly, there is a clear, on-going, and urgent need to reduce time and accuracy of diagnosis of hematopoietic malignancies through methods that permit simultaneous culturing of all four cell lineages or that permit the flexibility of rapidly transitioning from one cell lineage to another.

### SUMMARY

Described herein in part is a culture medium for culturing cells to assess cell lineages (e.g., B-cells, T-cells, plasma cells, and myeloid cells) from a specimen of a subject. The present disclosure is based, in part, upon the discovery of an in vitro cell culture medium, that can selectively grow a plurality of different hematopoietic cell lineages, that is, for example, dependent on the incubation time period. For example, provided herein is a culture medium comprising: a base medium; interleukin-2; lipopolysaccharide; one, two or more antibiotics; animal serum; and a growth factor. Disclosed culture mediums may optionally include glutamine.

The disclosure also contemplates a method of culturing bone marrow, peripheral blood and/or hematopoietic cells comprising contacting the cells with disclosed culture medium to form a culture; and incubating the culture for a set period of time.

The disclosure further contemplates a method of testing for a hematopoietic disease type comprising contacting a patient's cell specimen (e.g., a bone marrow biopsy) with disclosed culture medium to form a first and second culture; and incubating the cultures for a set period of time, determining a further incubation period of the second culture based on a clinical indication informed by the harvesting of the first culture and/or flow cytometry analysis of the patient's cells and continuing incubation of the second culture for the further incubation period, and determining the diagnosis of the hematopoietic disease based on analyzing the second culture after the further incubation period. Disclosed methods of testing for a hematopoietic disease type may optionally comprise determining an amount of cell specimen (for example, by cell counting) needed for culturing relative to a volume of the culture medium.

### BRIEF DESCRIPTION OF THE DRAWINGS

FIG. 1A is a flowchart depicting a typical cell culture workflow to diagnose hematopoietic disease, requiring selection of cell lineage at day 1. FIG. 1B is a flowchart depicting the workflow to diagnose hematopoietic disease using the culture medium of Example 1, allowing selection of cell lineage at day 3 so that additional information can be used to arrive at the selection. FIG. 1C is a table of incubation times for culturing various hematopoietic cell lineages in the culture medium of Example 1. C1 represents a first culture C1 incubated for 24 hours and C2 represents a second culture 2.

FIG. 2 is a photomicrograph showing chromosomes prepared from cells cultured in Karyo-Max™ supplemented medium or culture medium described in Example 1, side by side, for comparison of chromosome banding resolution.

FIG. 3 is a photomicrograph showing chromosomes prepared from myeloid cells cultured in Karyo-Max™ supplemented medium or the culture medium of Example 1.

FIG. 4 is a photomicrograph showing chromosomes prepared from B-cells cultured in Karyo-Max™ supplemented medium or the culture medium of Example 1.

FIG. 5 is a photomicrograph showing chromosomes prepared from plasma cells cultured in Karyo-Max™ supplemented medium or the culture medium of Example 1.

FIG. 6 is a table summarizing the results of a parallel study comparing the chromosome banding resolutions and abnormalities detected for cells cultured in House Brewed Medium, MarrowMax medium or medium of Example 1. HBM—House Brewed Medium; MX—MarrowMax medium; Ex 1—Example 1 medium; C1—24 hour unstimulated culture; C2—48 hour unstimulated culture; CB—96 hour unstimulated culture; LPS—lipopolysaccharides; IL2—Interleukin-2; CML—chronic myelogenous leukemia; CLL—chronic lymphocytic leukemia; MDS—Myelodysplastic syndrome; MGUS—monoclonal gammopathy of undetermined significance which is characterized by poor growth during culturing.

### DETAILED DESCRIPTION

The present disclosure is based in part, upon the discovery of an in vitro cell culture. The cell culture makes possible to cell culture for different hematopoietic cell lineages in the disclosed culture medium and for example, can postpone selection of cell lineages for e.g., 24 hours or more without the need for supplementation with specific mitogens.

Provided herein, in an embodiment, is a culture medium for simultaneously culturing cells from e.g., a plurality of cell lineages (e.g., B-cells, T-cells, plasma cells, and myeloid cells) from a bone marrow or peripheral blood specimen of a patient or subject.

### Definitions

The following definitions are included for the purpose of understanding the present subject matter and for constructing the appended patent claims.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” or “the,” includes the plural (and vice versa) unless specifically stated otherwise.

Where the use of the term “about” is before a quantitative value, the present teachings also include the specific quantitative value itself, unless specifically stated otherwise. As used herein, the term “about” refers to a ±15% variation from the nominal value unless otherwise indicated or inferred.

It should be understood that the order of steps or order for performing certain actions is immaterial so long as the present teachings remain operable. Moreover, two or more steps or actions can be conducted simultaneously.

At various places in the present specification, values are disclosed in groups or in ranges. It is specifically intended that the description include each and every individual subcombination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 1, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the disclosure unless claimed. No language in the specification should be construed as indicating any non-claimed element as essential to the practice of the present teachings.

As used herein, “patient” refers to a mammal, such as a human.

As used herein, a “medium” or “cell culture medium or media” refers to an aqueous based solution that provides for the growth, viability, or storage of cells and can for example promote the desired cellular activity, such as cell viability, growth, proliferation, differentiation of the cells cultured in the media.

As used herein a “base media” or “base medium” refers to a basal salt nutrient or an aqueous solution of salts and other elements that provide cells with water and certain bulk inorganic ions essential for normal cell metabolism and maintains intra- and extra-cellular osmotic balance. In various embodiments, a base media comprises at least one carbohydrate as an energy source, and/or a buffering system to maintain the medium within the physiological pH range. Examples of commercially available basal media include, but are not limited to, phosphate buffered saline (PBS), Dulbecco's Modified Eagle's Medium (DMEM), Minimal Essential Medium (MEM), Basal Medium Eagle (BME), RPMI 1640, Ham's F-10, Ham's F-12, α-Minimal Essential Medium (aMEM), Glasgow's Minimal Essential Medium (G-MEM), and Iscove's Modified Dulbecco's Medium. A base medium can be supplemented with nutrients, proteins, and growth factors.

### Culture Medium

The disclosure provides, at least in part, a culture medium comprising at least three or more components each selected from the group consisting of a base medium, interleukin-2; lipopolysaccharide; one, two or more antibiotics; animal serum; and/or a growth factor. For example, in certain embodiments, a disclosed culture medium comprises interleukin-2; lipopolysaccharide; one, two or more antibiotics; animal serum; and/or a growth factor, and may further comprise glutamine. In certain embodiments, a disclosed culture medium comprises interleukin-2; lipopolysaccharide; one, two or more antibiotics; animal serum; and/or a growth factor, and/or glutamine and/or RPMI 1640.

In certain embodiments, the lipopolysaccharide of a disclosed cell culture medium is a lipopolysaccharide derived from gram negative bacteria. Exemplary gram negative bacteria from which the lipopolysaccharide can be derived include Escherichia coli (E. coli), Klebsiella pneumonia, Pseudomonas aeruginosa, Salmonella minnesota, Salmonella typhimurium, Salmonella typhosa, and Serratia marcescens. In certain embodiments, the lipopolysaccharide is a lipopolysaccharide derived from the group of E. coli strains including E. coli 026:B6, E. coli 055:B5, E. coli 0111:B4, E. coli 0127:B8, and E. coli 0128:B12. In certain embodiments, the lipopolysaccharide is a lipopolysaccharide from E. coli 055:B5.

In certain embodiments, a disclosed culture medium comprises about 0.01 to about 0.2 ng/ml lipopolysaccharide. In certain embodiments, the culture medium comprises about 0.01 to about 0.02 ng/ml, 0.03 to about 0.04 ng/ml, 0.05 to about 0.06 ng/ml, 0.07 to about 0.08 ng/ml, 0.09 to about 0.1 ng/ml, 0.11 to about 0.12 ng/ml, 0.13 to about 0.14 ng/ml, 0.15 to about 0.16 ng/ml, 0.17 to about 0.18 ng/ml, or 0.19 to about 0.2 ng/ml of lipopolysaccharide. In certain embodiments, the culture medium comprises about 0.5 ng/ml, about 0.4 ng/ml, about 0.3 ng/ml, about 0.2 ng/ml, or about 0.1 ng/ml lipopolysaccharide. In certain embodiments, the culture medium comprises about 0.2 ng/ml lipopolysaccharide. In certain embodiments, the culture medium comprises about 0.01 ng/ml lipopolysaccharide.

In certain embodiments, the culture medium comprises an amount of lipopolysaccharide corresponding to a specific activity of about 1×10⁶ units/mg. For example, in certain embodiments, the culture medium comprises an amount of lipopolysaccharide corresponding to a specific activity of about 0.5×10⁶ units/mg, about 1×10⁶ units/mg, about 2×10⁶ units/mg, about 3×10⁶ units/mg, about 4×10⁶ units/mg, about 5×10⁶ units/mg, about 6×10⁶ units/mg, about 7×10⁶ units/mg, about 8×10⁶ units/mg, about 9×10⁶ units/mg, or about 1×10⁶ units/mg.

In certain embodiments, a disclosed cell culture medium includes interleukin-2, e.g., recombinant mouse IL-2. In certain embodiments, a disclosed cell culture medium includes interleukin-2 that is selected from the group comprising recombinant human IL-2, recombinant rhesus IL-2, recombinant mouse IL-2, recombinant rat IL-2, and recombinant porcine IL-2.

In certain embodiments, a disclosed culture medium comprises about 0.01 to about 0.2 ng/ml IL-2. In certain embodiments, a disclosed culture medium comprises about 0.01 to about 0.02 ng/ml, 0.03 to about 0.04 ng/ml, 0.05 to about 0.06 ng/ml, 0.07 to about 0.08 ng/ml, 0.09 to about 0.1 ng/ml, 0.11 to about 0.12 ng/ml, 0.13 to about 0.14 ng/ml, 0.15 to about 0.16 ng/ml, 0.17 to about 0.18 ng/ml, or 0.19 to about 0.2 ng/ml IL-2. In certain embodiments, a disclosed culture medium comprises about 0.5 ng/ml, about 0.4 ng/ml, about 0.3 ng/ml, about 0.2 ng/ml, or about 0.1 ng/ml IL-2. In certain embodiments, the culture medium comprises about 0.2 ng/ml IL-2. In certain embodiments, the culture medium comprises about 0.01 ng/ml IL-2.

In certain embodiments, a disclosed culture medium comprises for example, an amount of IL-2 corresponding to a specific activity of about 5×10⁶ units/mg. For example, in certain embodiments, the culture medium comprises an amount of IL-2 corresponding to a specific activity of about 0.5×10⁶ units/mg, about 1×10⁶ units/mg, about 2×10⁶ units/mg, about 3×10⁶ units/mg, about 4×10⁶ units/mg, about 5×10⁶ units/mg, about 6×10⁶ units/mg, about 7×10⁶ units/mg, about 8×10⁶ units/mg, about 9×10⁶ units/mg, or about 1×10⁶ units/mg.

In certain embodiments, a disclosed culture medium comprises one, two or more antibiotics to prevent contamination of the culture by microorganisms. Exemplary antibiotics suitable for use in the culture medium include penicillin, streptomycin, gentamicin, kanamycin, neomycin, ampicillin, carbenicillin, and cefotaxime. In certain embodiments, the antibiotics are each independently selected from the group consisting of penicillin, streptomycin, and combinations thereof.

For example, a disclosed culture medium may include penicillin and streptomycin. The culture medium may, for example, comprise about 20 units/ml of an antibiotic or of each antibiotic. For example, in certain embodiments, the culture medium comprises about 20 units/ml penicillin. The culture medium may comprise about 2 units/ml, about 4 units/ml, about 6 units/ml, about 8 units/ml, about 10 units/ml, about 12 units/ml, about 14 units/ml, about 16 units/ml, about 18 units/ml, about 20 units/ml, about 22 units/ml, about 24 units/ml, about 26 units/ml, about 28 units/ml, or about 30 units/ml penicillin. In other embodiments, the culture medium comprises about 20 μg/ml of an antibiotic or of each antibiotic. For example, the culture medium comprises about 20 μg/ml streptomycin. The culture medium may comprise about 2 μg/ml, about 4 μg/ml, about 6 μg/ml, about 8 μg/ml, about 10 μg/ml, about 12 μg/ml, about 14 μg/ml, about 16 μg/ml, about 18 μg/ml, about 20 μg/ml, about 22 μg/ml, about 24 μg/ml, about 26 μg/ml, about 28 μg/ml, or about 30 μg/ml streptomycin. In some embodiments, the culture medium comprises about 20 units/ml penicillin and about 20 μg/ml streptomycin.

A disclosed culture medium includes a based medium, e.g., RPMI 1640 as a base medium. RPMI 1640 Medium contains biotin, vitamin B₁₂, and PABA, and the vitamins inositol and choline, which are present in very high concentrations. RPMI 1640 Medium contains no proteins, lipids, or growth factors. The complete formulation of RPMI 1640 is listed in table 1.

TABLE 1
RPMI 1640 formulation

| | | mg/l, final |
|---|---|---|
| | Composition | medium |
| Inorganic salts | Calcium chloride × 2H2O | 62.27 |
| | Potassium chloride | 400 |
| | Magnesium sulfate dried | 69.77 |
| | Sodium chloride | 5950.49 |
| | Sodium nitrate | 72 |
| | di-Sodium hydrogen phosphate anhydr. | 800 |
| Other Components | D(+)-Glucose anhydr. | 2000 |
| | L-Glutathione red. | 1 |
| Amino acids | L-Arginine HCl | 241.86 |
| | L-Asparagine × H2O | 50 |
| | L-Aspartic acid | 20 |
| | L-Cystine | 50 |
| | L-Glutamic acid | 20 |
| | Glycine | 10 |
| | L-Histidine base | 15 |
| | L-Hydroxyproline | 20 |
| | L-Isoleucine | 50 |
| | L-Leucine | 50 |
| | L-Lysine × HCl | 40 |
| | L-Methionine | 15 |
| | L-Phenylalanine | 15 |
| | L-Proline | 20 |
| | L-Serine (non animal origin) | 30 |
| | L-Threonine | 20 |
| | L-Tryptophan | 5 |
| | L-Tyrosine | 20 |
| | L-Valine | 20 |
| Vitamins | 4-Amino benzoic acid | 1 |
| | D(+)-Biotin | 0.2 |
| | D-Calcium pantothenate | 0.25 |
| | Choline chloride | 3 |
| | Folic acid | 1 |
| | myo-Inositol | 35 |
| | Nicotinamide | 1 |
| | Pyridoxine × HCl | 1 |
| | Riboflavin | 0.2 |
| | Thiamine × HCl | 1 |
| | Vitamin B12 | 0.005 |

In certain embodiments, a disclosed culture medium is supplemented with animal serum which provides proteins, nutrients, attachment factors, trace elements, growth factors, and hormones, which aid in the growth of the cells. It should be appreciated that although fetal bovine serum (FBS) is the most commonly used serum product, other available serum alternatives are contemplated. Exemplary serum products suitable for use in the cell culture medium include rabbit serum, goat serum, lamb serum, porcine serum, horse serum, chicken serum, and bovine serum (e.g., fetal bovine serum and newborn calf serum).

For example, in certain embodiments, the culture medium includes animal serum which is bovine serum, e.g., fetal bovine serum. A disclosed culture medium may include an amount of animal serum corresponding to a specific activity of about 1×10⁶ units/mg. For example, in certain embodiments, the culture medium comprises an amount of fetal bovine serum corresponding to a specific activity of about 1×10⁶ units/mg. In certain embodiments, the culture medium comprises an amount of fetal bovine serum corresponding to a specific activity of about 0.5×10⁶ units/mg, about 1×10⁶ units/mg, about 2×10⁶ units/mg, about 3×10⁶ units/mg, about 4×10⁶ units/mg, about 5×10⁶ units/mg, about 6×10⁶ units/mg, about 7×10⁶ units/mg, about 8×10⁶ units/mg, about 9×10⁶ units/mg, or about 1×10⁷ units/mg.

In certain embodiments, a disclosed culture medium comprises glutamine (e.g., L-glutamine). Glutamine participates in the formation of purine and pyrimidine nucleotides, amino sugars, glutathione, L-glutamate, other amino acids, and is used in protein synthesis and glucose production. Glutamine is one of the most readily available amino acids for use as an energy source and it is a major source of energy for many rapidly dividing cell types in vitro.

In certain embodiments, the culture medium comprises about 1 to about 20 mM glutamine. For example, in certain embodiments, the culture medium may comprise about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM glutamine.

In other embodiments, the culture medium comprises an amount of glutamine corresponding to a specific activity of about 1×10⁶ units/mg. For example, in certain embodiments, the culture medium comprises an amount of glutamine corresponding to a specific activity of about 0.5×10⁶ units/mg, about 1×10⁶ units/mg, about 2×10⁶ units/mg, about 3×10⁶ units/mg, about 4×10⁶ units/mg, about 5×10⁶ units/mg, about 6×10⁶ units/mg, about 7×10⁶ units/mg, about 8×10⁶ units/mg, about 9×10⁶ units/mg, or about 1×10⁷ units/mg.

In certain embodiments, the culture medium of the present disclosure comprises a growth factor, for example, giant cell tumor promotor (GCT). It should be appreciated that GCT which is harvested from a cultured giant cell tumor cell line (derived from a human malignant fibrous histiocytoma) is a potent source of the colony stimulating factors for the growth of hematopoietic progenitor cells from human, mouse or rabbit bone marrow or peripheral blood. In certain embodiments, the culture medium comprises an amount of giant cell tumor promotor corresponding to a volume percent of about 1% v/v to about 20% v/v. The culture medium may comprise an amount of giant cell tumor promotor corresponding to a volume percent of about 1% v/v, about 5% v/v, about 10% v/v, about 15% v/v, or about 20% v/v. In certain embodiments, the culture medium comprises an amount of giant cell tumor promotor corresponding to a volume percent of about 5% V/V.

For example, disclosed herein is a cell medium culture comprising: RPMI 1640 as a basic cell medium (e.g., with no glutamine added); about 0.1 to about 0.3 ng/mL IL-2 (e.g., mouse recombinant IL-2 expressed in E. coli and in the form of e.g., lyophilized powder); about 0.1 to about 0.3 ng/ml lipopolysaccharides (e.g., from Escherichia coli O55:B); about 10 to about 30 units/mL of penicillin; about 10 to about 30 μg/mL of streptomycin; fetal bovine serum in an amount that provides a specific activity of about 1×10⁶ units/mg; about 10 to about 30 mM of glutamine and about 3 to about 6% V/V giant cell tumor promotor.

For example, provided herein is a cell medium culture comprising: a basic cell medium (e.g., RPMI 1640 (e.g., with no glutamine added)); about less than or equal to 0.2 ng/mL IL-2 (e.g., mouse recombinant IL-2 expressed in E. coli and in the form of e.g., lyophilized powder, e.g., about 0.1 to about 0.2 ng/mL); about 0.2 ng/ml lipopolysaccharides (e.g., from Escherichia coli O55:B); penicillin and/or streptomycin (e.g., a penicillin/streptomycin solution that provides about 20 units/mL of penicillin and about 20 μg/mL of streptomycin); an amount of fetal bovine serum that provides a specific activity of 1×10⁶ units/mg; about 20 mM of glutamine and about 5% V/V giant cell tumor promotor.

### Cell Culture

In another aspect, the present disclosure contemplates a method of simultaneously culturing a plurality of different cell lineages comprising culturing the plurality of cells in the culture medium. In some embodiments, the plurality of cells are first obtained from a patient's specimen, e.g., a bone marrow specimen or a peripheral blood specimen.

In an embodiment, provided herein is a method of simultaneously culturing a plurality of different cell lineages comprising culturing the plurality of cells in the culture medium wherein the patient's cells are from a bone-marrow biopsy. Exemplary cell types that can be grown in the culture medium include hematopoietic stem cells, myeloid progenitors, megakaryocytes, erythrocytes, mast cells, myeloblasts, lymphoid progenitors, natural killer cells, T-cells, B-cells, and plasma cells. In an embodiment, provided herein is a method of simultaneously culturing a plurality of different cell lineages which are B-cells, T-cells, plasma cells, and myeloid cells.

For example, in certain embodiments, provided herein is a method of culturing bone marrow, peripheral blood and/or hematopoietic cells comprising: contacting the cells with a culture medium of the disclosure to form a culture; and incubating the culture for a set period of time. In some embodiments, the set period of time is selected from an incubation time corresponding to different cell lineages selected from the group consisting of B-cells, T-cells, plasma cells, and myeloid cells. In some embodiments of the disclosed method, the set period of time is determined at least 4 hours after the contacting step.

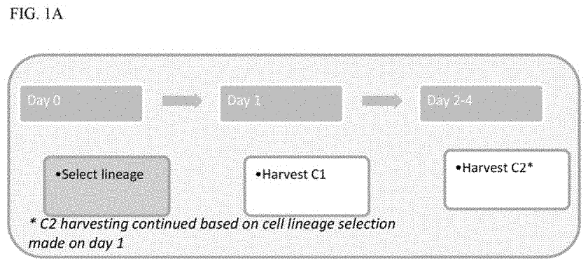

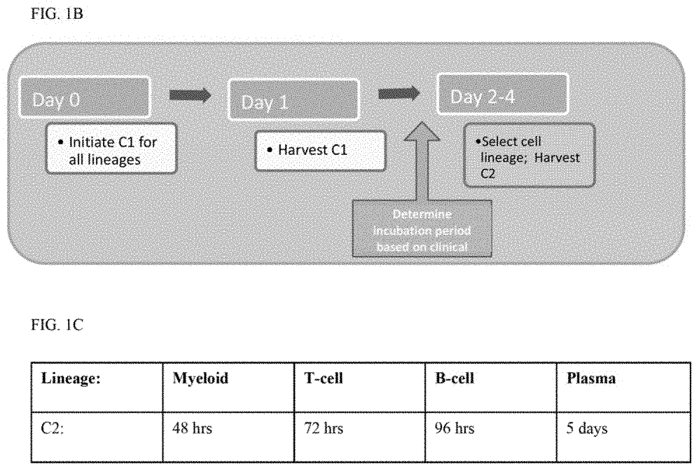

In some embodiments, provided herein and schematically summarized in FIG. 1B, are methods of testing for a hematopoietic disease type, comprising: contacting a patient's cell specimen with the culture medium to form a first and second culture; incubating the first and second culture for about 24 hours; harvesting the first culture; determining a further incubation period of the second culture based on clinical indication informed by the harvesting of the first culture and/or flow cytometry analysis of the patient's cells; continuing incubation of the second culture for the further incubation period; and determining the diagnosis of the hematopoietic disease based on analyzing the second culture after the further incubation period. In some embodiments, the patient's cells are from a bone-marrow biopsy.

In some embodiments of disclosed methods of testing for a hematopoietic disease type, determining a further incubation period of the second culture is at least 4 hours after the second and first culture is formed. In some embodiments, determining the further incubation period comprises selecting an incubation period suitable for a cell selected from the group consisting of myeloid cells, B-cells, T-cells and plasma cells. For example, in some embodiments, the incubation periods suitable for growing cells (e.g., B-cells, T-cells and plasma cells) in the culture medium is provided in FIG. 1C.

In some embodiments, methods of testing for a hematopoietic disease type further comprises determining an amount of cell specimen needed for culturing relative to a volume of the culture medium. For example, in some embodiments, determining the amount of cell specimen needed comprises counting cells.

### EXAMPLES

In order that the disclosure described herein can be more fully understood, the following examples are set forth. It should be understood that these examples are included merely for purposes of illustration of certain aspects and embodiments of the present disclosure, and are not to be construed as limiting the disclosure in any manner.

### Example 1: Preparation of Media Used for Culturing Bone Marrow and Peripheral Blood Cells

The reagents listed in Table 2 are used:

TABLE 2
Example 1 media formulation

| Culture | | | |
|---|---|---|---|
| medium | Composition | Vendor | Catalogue # |
| Example 1 | RPMI 1640 without L-G | Thermo fisher | 21870-076 |
| | | Scientific | |
| | Fetal Bovine calf serum | Thermo fisher | 16000-036 |
| | | Scientific | |
| | L-Glutamine | Thermo fisher | 25030-081 |
| | | Scientific | |
| | Pen-strep | Thermo fisher | 15070-063 |
| | | Scientific | |
| | Lipopolysaccharides | Sigma | L2880-25MG |
| | Interleukin-2 | Sigma | I0523-20UG |

All reagents and Media were prepared following the reagent/media preparation protocols. To prepare media, the full LPS powder was dissolved in 25 ml sterile DW (distilled water) and the full IL-2 powder was carefully dissolved in 10 ml RPMI 1640 under the biological hood.

The following were then combined in the RPMI 1640 bottle: 500 ml RPMI 1640 without L-Glutamine, 100 ml bovine fetal calf serum and 10 ml of L-Glutamine. To this mixture, 5 ml of Pen-strep (penicillin-streptomycin) and 25 ml of the previously prepared LPS solution was added. The contents were mixed by inverting the bottle a couple of times. 10 ml of the previously prepared IL-2 solution was then added to the mixture, and the contents were mixed by inverting the bottle a couple of times.

The mixed media was then distributed into two bottles labeled as “A” and “B”. Media A was used for the first culture setup and media B was used with the second culture for subsequent experiments.

The reagents listed in Table 3 were selected for use in preparing the comparative home brewed (HBM) and MarrowMax (MX) media.

TABLE 3
Comparative Media reagents

| Culture medium | Composition | Vendor | Catalogue # | Quantity |
|---|---|---|---|---|
| HBM | RPMI 1640 without L-G | Thermo fisher Scientific | 21870-076 | 500 ml |
| | Fetal Bovine calf serum | Thermo fisher Scientific | 16000-036 | 100 ml |
| | L-Glutamine | Thermo fisher Scientific | 25030-081 | 10 ml |
| | Pen-strep | Thermo fisher Scientific | 15070-063 | 5 ml |
| MARROWMAX ™ | Fetal Bovine Serum (FBS) | Thermo fisher Scientific | 12260001 | |
| | Gentamicin | | | |
| | L-Glutamine | | | |
| | hematopoietic growth factors | | | |

To prepare home brewed media (HBM), fetal bovine calf serum, L-Glutamine and pen-strep were combined with RPMI 1640 without L-Glutamine under the biological hood. The contents were mixed by inverting the bottle a few times.

To prepare MarrowMax™ media (MX), fetal bovine serum, gentamicin, L-Glutamine and hematopoietic growth factors were combined with MarrowMax bone marrow medium under the biological hood. The contents were mixed by inverting the bottle a few times.

### Example 2: Examination of Bone Marrow and Peripheral Blood Cells for Chromosomal Abnormalities

Cell counts of bone marrow or peripheral blood specimen were performed using a Hemocytometer and Trypan blue staining. A working Trypan blue solution was prepared by mixing 95 mL dH₂O, 3 mL acetic acid and 2 mL Wright's stain. A 1:100 dilution of the specimen was made by adding 5 μl of specimen to 0.5 mL Trypan blue solution in Acetic acid in eppendorf tube. The suspension was vortexed and 5 μl of the mixture was applied to the Hemocytometer. After waiting for a few minutes for the cells to settle down on the slide, cells were counted in the squares of the hemocytometer using a microscope.

Table 4 below was used as a guide to determine the appropriate specimen amount needed for culturing relative to the medium volume. Unused specimens were kept in the refrigerator for two weeks.

TABLE 4

| | Specimen in μl per 10 mL |
|---|---|
| Count @ 1:100 dilution | medium |
| 10 | 1200 |
| 20 | 700 |
| 30 | 500 |
| 40 | 300 |
| 50 | 300 |
| 60 | 200 |
| 70 | 200 |
| 80 | 200 |
| 90 | 150 |
| 100 or >100 | 150 |

The bone marrow or leukemic peripheral blood cells were cultured in duplicate according to the incubation periods indicated below in Table 5 for the first and second culture for each cell type. All cultures were grown in 10 ml media conical tubes. Each culture tube was appropriately labeled with at least two unique identifiers; requisition number and patient's name and the sub-culture number.

TABLE 5
Incubation period for various indications

| Culture | Myeloid disorders | T cell disorders | B cell disorders |
|---|---|---|---|
| C1 | 18-24 Hours | 18-24 Hours | 18-24 Hours |
| C2 | 48 hours | None | None |
| C3 | None | 72 hours | None |
| C4 | None | None | 96-120 hours |

The specimen tube was carefully opened under the hood using gauze. By using a sterile transfer pipette, the specimen was gently mixed and the required amount of the specimen was added to the culture tube. Cell cultures were maintained at 37° C., 5% CO₂ in a humidified incubator. Images for chromosomal diagnostics analysis was captured using CytoVision Automated Cytogenetics Platform.

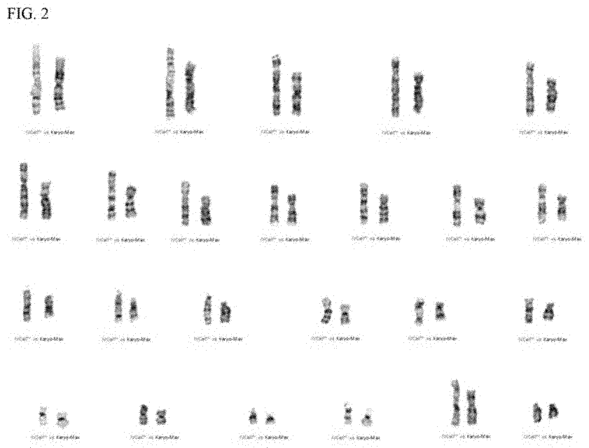

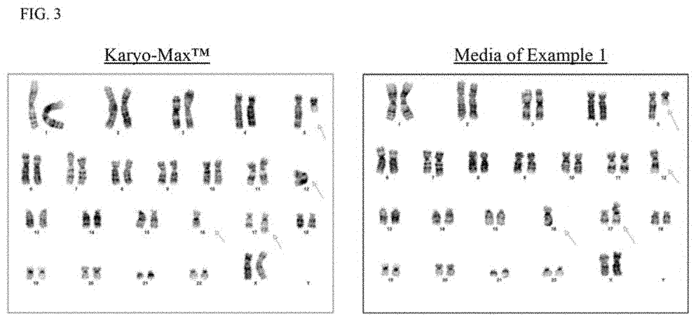

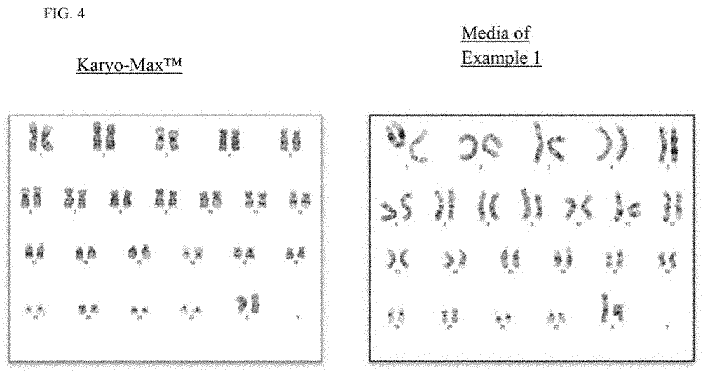

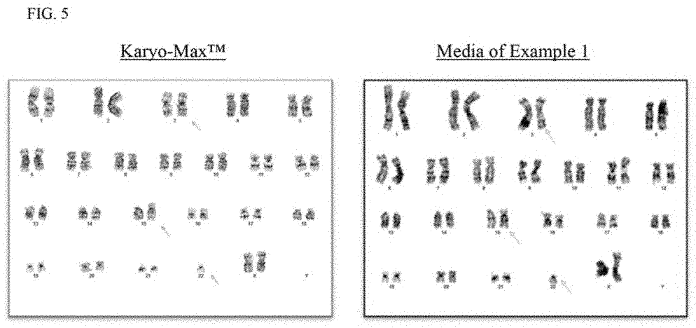

Cytogenetic results demonstrated that cells cultured in Example 1 media consistently showed higher resolution of chromosomal banding patterns (allowing for easier identification of chromosomal aberrations) compared to other culture media. As shown in FIG. 2, upon culturing with Example 1 media, the average chromosomal band count was about 500-500. This higher resolution was true for multiple cell lineages or indications including myeloid cells (FIG. 3), B-cells (FIG. 4) and plasma cells (FIG. 5).

### Example 3: Chromosomal Band Resolution and Detection of Chromosomal Abnormalities of Cells Cultured in Different Media

The objective of this experiment was to perform a parallel study to compare the performance of Example 1 culture media against other media reagents existing in the market for examination of chromosomal bands and abnormalities for diagnostic and prognostic information on hematological malignancies.

Cell counts of bone marrow or peripheral blood specimen from patient samples were performed using a Hemocytometer and Trypan blue staining. A working Trypan blue solution was prepared by mixing 95 mL dH₂O, 3 mL acetic acid and 2 mL Wright's stain. A 1:100 dilution of the specimen was made by adding 5 μl of specimen to 0.5 mL Trypan blue solution in Acetic acid in eppendorf tube. The suspension was vortexed and 5 μl of the mixture was applied to the Hemocytometer. After waiting for a few minutes for the cells to settle down on the slide, cells were counted in the squares of the hemocytometer using a microscope.

Table 4 was used as a guide to determine the appropriate specimen amount needed for culturing relative to the medium volume. Unused specimens were kept in the refrigerator for two weeks.

Specimen cultures were set up in duplicate. All cultures were grown in 10 ml media conical tubes. Each culture tube was appropriately labeled with at least two unique identifiers; requisition number and patient's name and the sub-culture number.

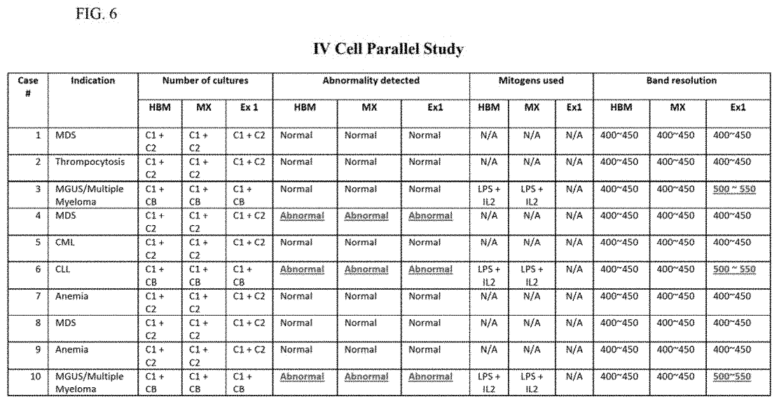

The specimen tube was carefully opened under the hood using gauze. By using a sterile transfer pipette, the specimen was gently mixed and the required amount of the specimen was added to the culture tube. Cell cultures were maintained at 37° C., 5% CO₂ in a humidified incubator according to the incubation periods indicated in FIG. 6.

Chromosomal images were prepared as follows:

### Sample Processing (Harvesting Procedure)

1. Add 8 uL EB “Ethidium bromide” per 1 mL of culture, incubate at 37° C. for 45 minutes
2. Add 10 uL Colcemid per 1 mL of culture; incubate at 37° C. for 30 min.
3. Centrifuge for 10 min at 1000 rpm.
4. Aspirate the supernatant carefully leaving around 0.5 mL supernatant above the pellet.
5. Gently mix the pellet by tapping the tube then add up to 12 mL hypotonic solution (previously wormed at 37° C.), gently mix well, incubate at 37° C. for 16-25 min
6. Add 2 mL freshly prepared fixative slowly, gently mix well, leave at Room Temperature for 10 min, then centrifuge for 10 min at 1000 rpm.
7. Aspirate the supernatant carefully leaving around 0.5 mL supernatant above the pellet.
8. Add 10 mL fixative slowly, gently mix the pellet, leave at Room Temperature for 10 min, then centrifuge for 10 min at 1000 rpm.
9. Aspirate the supernatant carefully, add 5-7 mL fixative (depending on the pellet size), gently mix well, centrifuge for 10 min at 1000 rpm.
10. Aspirate the supernatant carefully, add 3-5 mL fixative (depending on the pellet size), gently mix well, centrifuge for 10 min at 1000 rpm.
11. It is preferred leaving the pellet in the refrigerator for 30 min before dropping for better quality banding, or store the pellet in the refrigerator for later use.
12. Save all pellets in fridge for 6 months after case submission. Discard all pellets after 6 months unless noted differently by the cytogenetics supervisor.

### Slide Preparation

1. Spin the cell suspension for 10 minutes at 1000 rpm and aspirate the suspension as before and then re-suspend pellet “should be white at this stage” in a small volume of fixative 0.5-1 mL depending on pellet size “should appear cloudy”. Evaluate slides made: More fix can be added if too concentrated or spin down & re-suspend in smaller volume if too diluted. Use a new transfer pipette for each culture tube.
2. Drop slides from one case at a time.
3. Place a paper towel flat on the counter top make it wet by spraying water on it. Keep the towel wet through out the dropping procedure.
4. Use a clean slide from the slide box, dip it into water at room temperature.
5. While holding the slide at a slight angle drop a small amount of the cell suspension on it.
6. The slide is placed on the humid wet towel for 50-90 seconds (depending on the room humidity).
7. Wipe the back of the slide, and the slide is immediately placed on a warm plate (40-46° C.) until completely dry.
8. Prepare 3-4 slides per culture or as needed.

### Aging of Slides:

Place the slides in the oven at 90° C. for 60 minutes.

### Staining Procedure:

Set up 6 staining jars proceed staining as follows:
1. Jar 1: mix 40 mL “Balanced Salt pH 7.0” & 5 mL Trypsin 25-60 sec
2. Jar 2: 40 mL “Balanced Salt pH 7.0” dip twice
3. Jar 3: 40 mL “Balanced Salt pH 7.0” dip twice
4. Jar 4: mix 40 mL “Gurr Buffer pH 6.8” & 2 mL Giemsa 45-90 sec
5. Jar 5: 40 mL “Distilled Water” dip twice
6. Jar 6:40 mL “Distilled Water” dip twice
   Staining duration of 10 second for Giemsa and 5 seconds for Trypsin should be increased after staining of 10-15 slides. Air dry slides in a slanting position or using slide warmer. The slides are ready for scanning, slides will be stored at room temperature until the analysis begins. Capturing and karyotyping are conducted per the imaging system (Cytovision).

Cytogenetic results demonstrated that Example 1 media consistently performed as well as other media reagents on the market in the ability to detect chromosomal abnormalities (FIG. 6). Furthermore, as shown in FIG. 6, culturing in Example 1 media yielded higher chromosomal banding resolutions for MGUS/Multiple Myeloma and CLL patients as compared to other media (House brewed and MarrowMax media).

What is claimed is:

1. A culture medium comprising:
a base medium;
interleukin-2;
lipopolysaccharide;
one, two or more antibiotics;
animal serum; and
a growth factor.

2. The culture medium of claim 1, further comprising glutamine.

3. The culture medium of claim 1, wherein the lipopolysaccharide is lipopolysaccharide from E. coli 055:B5.

4. The culture medium of claim 1, wherein the interleukin-2 is recombinant mouse IL-2.

5. The culture medium of claim 1, wherein the antibiotics are each independently selected from the group consisting of penicillin, streptomycin, and combinations thereof.

6. The culture medium of claim 1, wherein the animal serum is bovine serum.

7. The culture medium of claim 1, wherein the animal serum is fetal bovine serum.

8. The culture medium of claim 1, wherein the growth factor is giant cell tumor promotor.

9. The culture medium of claim 1, comprising about 0.01 to about 0.2 ng/ml IL-2.

10. The culture medium of claim 1, comprising an amount of IL-2 corresponding to a specific activity of about 5×10⁶ units/mg.

11. The culture medium of claim 1, comprising about 0.01 to about 0.2 ng/ml lipopolysaccharide.

12. The culture medium of claim 1, comprising an amount of lipopolysaccharide corresponding to a specific activity of about 1×10⁶ units/mg.

13. The culture medium of claim 1, comprising an amount of animal serum corresponding to a specific activity of about 1×10⁶ units/mg.

14. The culture medium of claim 1, comprising about 1 to about 20 mM glutamine.

15. The culture medium of claim 1, comprising an amount of glutamine corresponding to a specific activity of about 1×10⁶ units/mg.

16. The culture medium of claim 1, comprising an amount of giant cell tumor promotor corresponding to a volume percent of about 5% v/v.

17. A cell culture medium comprising:
RPMI 1640;
about 0.1 to about 0.3 ng/mL IL-2;
about 0.1 to about 0.3 ng/ml lipopolysaccharides;
about 10 to about 30 units/mL of penicillin;
about 10 to about 30 μg/mL of streptomycin;
fetal bovine serum in an amount that provides a specific activity of about 1×10⁶ units/mg;
about 10 to about 30 mM of glutamine and
about 3 to about 6% V/V giant cell tumor promotor.

18. A method of culturing bone marrow, peripheral blood and/or hematopoietic cells comprising:
contacting the cells with a culture medium of claim 1 to form a culture; and
incubating the culture for a set period of time.

19. The method of claim 18, wherein the set period of time is selected from an incubation time corresponding to different cell lineages selected from the group consisting of: B-cells, T-cells, plasma cells, and myeloid cells.

20. The method of claim 18, wherein the set period of time is determined at least 4 hours after the contacting step.

21. A method of testing for a hematopoietic disease type, comprising:
contacting a patient's cell specimen with the culture medium of claim 1 to form a first and second culture;
incubating the first and second culture for about 24 hours;
harvesting the first culture;
determining a further incubation period of the second culture based on a clinical indication informed by the harvesting of the first culture and/or flow cytometry analysis of the patient's cells and continuing incubation of the second culture for the further incubation period;
determining the diagnosis of the hematopoietic disease based on analyzing the second culture after the further incubation period.

22. The method of claim 21, wherein the determining the further incubation period comprises selecting an incubation period suitable for a cell selected from the group consisting of: myeloid cells, B-cells, T-cells and plasma cells.

23. The method of claim 21, wherein the patient's cells are from a bone-marrow biopsy.

24. The method of claim 21, wherein determining a further incubation period of the second culture is at least 4 hours after the second and first culture is formed.

25. The method of claim 21, further comprising determining an amount of cell specimen needed for culturing relative to a volume of the culture medium.
